(19) United States
(12) Patent Application Publication
(10) Pub. No.: US 2020/0390705 A1
(43) Pub. Date: Dec. 17, 2020

(54) METHOD FOR REDUCING THE RECONSTITUTION TIME OF SPRAY-DRIED PROTEIN FORMULATIONS

(71) Applicant: UCB BIOPHARMA SRL, Brussels (BE)

(72) Inventors: Maarten BATENS, Slough, Berkshire (GB); Jan Ivo MASSANT, Brussels (BE)

(21) Appl. No.: 16/764,338

(22) PCT Filed: Nov. 13, 2018
(86) PCT No.: PCT/EP2018/081058

§ 371 Date: May 14, 2020

(30) Foreign Application Priority Data
Nov. 15, 2017 (GB) 1718888.9

(51) Int. Cl. A61K 9/16; A61K 39/395

(52) U.S. Cl. CPC A61K 9/1617; A61K 39/39591; A61K 9/1623

(57) ABSTRACT

The present invention relates to the use of a combination of sugar and one or more amino acids for reducing the reconstitution time of a spray-dried protein formulation.

### FIELD OF THE INVENTION

The present invention belongs to the field of spray-dried pharmaceutical formulations and their manufacturing processes. More specifically, it relates to methods, uses and processes for reducing the reconstitution time of spray-dried protein formulations for pharmaceutical uses.

### BACKGROUND OF THE INVENTION

Liquid injections of monoclonal antibodies such as intravenous, intramuscular or subcutaneous injections, are still the most preferred route of administration which enable to yield high systemic concentration of monoclonal antibodies for therapeutic uses (Wan et al. Technologies 9 (2), e141-e146. (2012); Roberts, C. J., Current Opinion in Biotechnology 30, 211-217 (2014); Moroz, E. et al. Advanced Drug Delivery Reviews 101, 108-121 (2016)). Subcutaneous injections are the preferred route of the aforementioned options as they allow the patient to self-administer, potentially limiting the impact on the patient's daily life and overall treatment cost. However, the volume that can be administered via subcutaneously in the absence of specialised devices or enzymatic methods like hyaluronidase-facilitated subcutaneous infusion systems, is very limited, around 1.5-2 ml, making it necessary to administer highly concentrated monoclonal antibody formulations (Wasserman, R. L., et al. Journal of Allergy and Clinical Immunology 130 (4), 951-957 (2012)). Consequently, the high concentrations of monoclonal antibodies in these formulations will increase the chances of protein self-interactions with an associated risk of irreversible aggregation, which leads to a decrease in product efficacy and an increased risk of immunogenic responses (Roberts, C. J., Current Opinion in Biotechnology 30, 211-217 (2014); Barnett et al. Biophysical Chemistry 207, 21-29 (2015); Mahler et al. Journal of Pharmaceutical Sciences 98(9), 2909-2934 (2009)).

Formulations of monoclonal antibodies for subcutaneous injections are often dried when their liquid form is insufficiently stable, as the removal of water drastically reduces protein conformational mobility and limits the transport of small-molecule reactants, subsequently reducing the rate of protein self-interaction and degradation mechanisms, thus improving the formulation's (storage) stability (Cicerone, M. T., et al. Soft Matter 8, 2983-2991 (2012)). Different mechanisms and models for protein stabilisation in liquid state, during drying and in solid state have been proposed over the years.

The drying of protein formulations is often done by lyophilisation, as there is no risk of exposing the proteins to high temperatures. However, a major downside to lyophilisation is the amount of stress generated both during the freezing and drying phases, potentially resulting in a partial or complete loss of activity.

Spray-drying of protein formulations, however, represents a fast, one-step, customizable process yielding powders having the desired morphology, density and powder flow. Although spray drying uses relatively high temperatures, the heat exposure of the protein is minimal as a result of heat being extracted from the droplet during solvent evaporation and the short duration of the spray drying process. To reduce the impact of stress factors like shear stress, exposure to air/liquid interfaces or increased heat stress upon loss of hydration shell and to further increase solid state stability, suitable excipients are added.

Spray-dried formulations, however, and similarly to other protein formulations in powder form, must be reconstituted, usually in water, just prior administration. Albeit reconstitution of a spray-dried formulation can easily be achieved, the time it occurs for this to be completed may widely vary depending on the excipients added to the protein formulation prior spray-drying. Considerable research has been devoted to determine the influence of these excipients on protein stability, and the majority has focused on lyophilisation or spray drying for inhalation, rather than on powders for subcutaneous injection. Furthermore, little is known about suitable excipients which may affect the reconstitution time for spray-dried protein formulations. Therefore, there remains a need in the art to provide further improved protein formulations for subcutaneous injections which once spray-dried reconstitute within an acceptable time.

### SUMMARY OF THE INVENTION

The present invention addresses the above-identified need by providing methods, processes and uses for reducing the reconstitution time of a spray-dried protein formulation through the combination of a sugar and one or more amino acids.

The following specific embodiments are described as numbered hereinafter:

### Embodiment 1

A method for reducing the reconstitution time of a spray-dried protein formulation, wherein the method comprises spray-drying a protein formulation comprising a protein in the presence of a sugar and one or more amino acids, wherein the sugar is a disaccharide and is present in an amount from 1.0 to 20% w/v and wherein the one or more amino acids is present in an amount from or from above 50 mM to 200 mM.

### Embodiment 2

The method according to Embodiment 1, wherein the protein is an antibody or a fragment thereof.

### Embodiment 3

The method according to Embodiment 1 or Embodiment 2, wherein the sugar is sucrose, trehalose or a mixture thereof.

### Embodiment 4

The method according to any one of the preceding embodiments wherein the one or more amino acids is glycine, L-proline, L-alanine, L-valine, L-serine, L-threonine, L-glutamine, L-asparagine, L-histidine, L-lysine, L-arginine or mixtures thereof.

### Embodiment 5

The method according to any one of the preceding embodiments wherein the sugar is sucrose or trehalose and the amino acid is L-arginine hydrochloride, L-histidine hydrochloride, L-lysine hydrochloride or mixtures thereof.

### Embodiment 6

The method according to any one of the preceding embodiments, wherein the method comprises spray-drying a protein formulation further comprising a surfactant.

### Embodiment 7

The method according to Embodiment 6, wherein the surfactant is a polysorbate, preferably polysorbate 20.

### Embodiment 8

A process for reducing the reconstitution time of a spray-dried protein formulation comprising the steps of:
a. Preparing a protein formulation comprising a protein, a sugar and one or more amino acids;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of a sugar and one or more amino acids, wherein the sugar is a disaccharide and is present in an amount from 1.0 to 20% w/v and wherein the one or more amino acids is present in an amount from or from above 50 mM to 200 mM.

### Embodiment 9

The process according to Embodiment 8, wherein the protein is an antibody or a fragment thereof.

### Embodiment 10

The process according to Embodiments 8 or 9, wherein the sugar is sucrose, trehalose or a mixture thereof.

### Embodiment 11

The process according to any one of Embodiments 8 to 10, wherein the amino acid is glycine, L-proline, L-alanine, L-valine, L-serine, L-threonine, L-glutamine, L-asparagine, L-glutamate, L-aspartate, L-histidine, L-lysine, L-arginine or mixtures thereof.

### Embodiment 12

The process according to any one of Embodiments 8 to 11, wherein the sugar is sucrose or trehalose and the amino acid is L-arginine hydrochloride, L-histidine hydrochloride, L-lysine hydrochloride or mixtures thereof.

### Embodiment 13

The process according to any one of Embodiments 8 to 12, wherein the method comprises spray-drying a protein formulation further comprising a surfactant.

### Embodiment 14

The process according to Embodiment 13, wherein the surfactant is a polysorbate, preferably polysorbate 20.

### Embodiment 15

A protein formulation obtained through the process according to any one of Embodiments 8 to 14.

### Embodiment 16

The protein formulation according to Embodiment 15 for use in therapy or diagnosis.

### BRIEF DESCRIPTION OF THE DRAWINGS

FIG. 1. Reconstitution time for formulations containing a sugar and combinations of amino acids. The effect of combination of amino acids was compared to 2.5% sucrose alone.

FIG. 2. Expanded parameter estimates plot of spray-dried formulations from DoE. The model was calculated using the values measured (n=3) for samples stored at 5° C. Open bars identify non-significant parameters, while parameters that are statistically significant at the 0.05 level are identified by shades bars. Error bars depict the estimated standard error for each of the estimated parameters. Intercept estimate=18.69±0.45 (SE) minutes.

FIG. 3. Reconstitution time of spray-dried formulations comprising mAb1. Reconstitution time comparison for formulations comprising selected amino acids and sucrose and a surfactant (black bars) versus a surfactant alone (white bars).

FIG. 4. Reconstitution times of spray-dried formulation comprising mAb1. Sucrose alone versus sucrose and single or combination of amino acids. No surfactant present.

FIG. 5. Reconstitution times of spray-dried formulation comprising mAb1. Sucrose and surfactant only versus sucrose, surfactant and single or combination of amino acids.

FIG. 6. Reconstitution times of spray-dried formulation comprising mAb1. Trehalose alone versus sucrose and single or combination of amino acids. No surfactant present.

FIG. 7. Reconstitution times of spray-dried formulation comprising mAb1. Trehalose and surfactant only versus sucrose, surfactant and single or combination of amino acids.

FIG. 8. Reconstitution times of spray-dried formulation comprising a Fab′-PEG antibody. The effect on the reconstitution time of a Fab′-PEG antibody formulation with sucrose alone versus sucrose in combination with increasing concentration of glycine.

FIG. 9. Reconstitution times of spray-dried formulation comprising a Fab′-PEG antibody. The effect on the reconstitution time of a Fab′-PEG antibody formulation with various amino acids at 1% in combination with 2.5% sucrose.

FIG. 10. Reconstitution times of spray-dried formulation comprising mAb1, a disaccharide and Arginine. A) Effect of trehalose whatever the concentration of Arginine. B) Effect of Arginine whatever the concentration of trehalose. C) Effect of the cumulative amount of Arginine and trehalose.

FIG. 11. Reconstitution times of spray-dried formulation comprising mAb1, a disaccharide and Glycine. A) Effect of trehalose whatever the concentration of Glycine. B) Effect of Glycine whatever the concentration of trehalose. C) Effect of the cumulative amount of Glycine and trehalose.

FIG. 12. Reconstitution times of spray-dried formulation comprising mAb1, a disaccharide and Lysine. A) Effect of trehalose whatever the concentration of Lysine. B) Effect of Lysine whatever the concentration of trehalose. C) Effect of the cumulative amount of Lysine and trehalose.

FIG. 13. Reconstitution times of spray-dried formulation comprising mAb1, a disaccharide and Proline. A) Effect of trehalose whatever the concentration of Proline. B) Effect of Proline whatever the concentration of trehalose. C) Effect of the cumulative amount of Proline and trehalose.

FIG. 14. Reconstitution times of spray-dried formulation comprising mAb2. The effect on the reconstitution time of mAb2 formulations with various amino acids at 100 mM in combination with 2.5% trehalose.

### DETAILED DESCRIPTION OF THE INVENTION

The method for reducing the reconstitution time of a spray-dried protein formulation according to the invention comprises spray-drying a protein formulation in the presence of a sugar and one or more amino acids.

Disaccharides and amino acids such as L-arginine have been reported to stabilize protein during lyophilisation and in solid state (Ohtake et al. Advanced Drug Delivery Reviews 63 (13), 1053-1073 (2011); Kamerzell et al. Advanced Drug Delivery Reviews 63 (13), 1118-1159 (2011) and Balcão and Vila, Advanced Drug Delivery Reviews 93, 25-41 (2015)). However, there have been no investigations on excipients and methods which may reduce the reconstitution time of a spray-dried protein formulation comprising a protein as described herein.

The present invention, therefore, also provides for the use of a combination of one or more amino acids and a sugar, such as a disaccharide, preferably sucrose or trehalose or a mixture thereof for reducing the reconstitution time of a spray-dried protein formulation.

In another aspect, the present invention also provides for a process for reducing the reconstitution time of a spray-dried protein formulation comprising the steps of:
a. Preparing a protein formulation comprising a protein, a sugar and one or more amino acids;
b. Spray-drying the protein formulation prepared in step a);
c. Recovering the spray-dried protein formulation of step b);
d. Reconstituting, preferably with water, the recovered spray-dried protein formulation within a reconstitution time RT1;
wherein the reconstitution time RT1 is less than the reconstitution time of the same protein formulation prepared in the absence of a sugar and one or more amino acids.

The term “in the presence of” as used herein means that the sugar and the one or more amino acids are part of the protein formulation before spray-drying that formulation and implies no limitation on how and when they have been added or the order of addition as long as the protein formulation before spray-drying contains them.

Acceptable reconstitution times are considered to be within 30 minutes, preferably within 25 minutes or more preferably within 20 minutes or earlier. The term “reconstitution time” as used herein means the time it takes to reconstitute a spray-dried protein formulation in a desired volume of solvent (e.g. water). Within the present disclosure, when considering whether certain excipients are unexpectedly superior than others, the reconstitution times for identically spray-dried protein formulations, but for the different excipients to be investigated, are compared. Unexpectedly superior excipients reduce the reconstitution time within these limits of at least about 10% or more.

Preferably, the protein in the protein formulation according to the method, use and process of the present invention is an antibody or antigen-binding fragment thereof.

The term “antibody” or “antibodies” as used herein refers to monoclonal or polyclonal antibodies and is not limited to recombinant antibodies that are generated by recombinant technologies as known in the art.

Preferably, the antibody comprised in the protein formulation according to the methods, uses and processes of the present invention is a monoclonal antibody.

“Antibody” or “antibodies” include antibodies' of any species, in particular of mammalian species, having two essentially complete heavy and two essentially complete light chains, human antibodies of any isotype, including IgA₁, IgA₂, IgD, IgG₁, IgG₂ₐ, IgG_{2b}, IgG₃, IgG₄ IgE and IgM and modified variants thereof, non-human primate antibodies, e.g. from chimpanzee, baboon, rhesus or cynomolgus monkey, rodent antibodies, e.g. from mouse, rat or rabbit; goat or horse antibodies, and derivatives thereof, or of bird species such as chicken antibodies or of fish species such as shark antibodies. The term “antibody” or “antibodies” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatised” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region or complementarity determining region (CDR) of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody performance. Humanization reduces the immunogenicity of non-human antibodies in humans, thus facilitating the application of antibodies to the treatment of human diseases. Humanized antibodies and several different technologies to generate them are well known in the art. The term “antibody” or “antibodies” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. For example, it has been described that the homozygous deletion of the antibody heavy-chain joining region (JH) gene in chimeric and germline mutant mice results in complete inhibition of endogenous antibody production. Transfer of the human germline immunoglobulin gene array in such germline mutant mice will result in the production of human antibodies with specificity against a particular antigen upon immunization of the transgenic animal carrying the human germline immunoglobulin genes with said antigen. Technologies for producing such transgenic animals and technologies for isolating and producing the human antibodies from such transgenic animals are known in the art. Alternatively, in the transgenic animal; e.g. mouse, only the immunoglobulin genes coding for the variable regions of the mouse antibody are replaced with corresponding human variable immunoglobulin gene sequences. The mouse germline immunoglobulin genes coding for the antibody constant regions remain unchanged. In this way, the antibody effector functions in the immune system of the transgenic mouse and consequently the B cell development are essentially unchanged, which may lead to an improved antibody response upon antigenic challenge in vivo. Once the genes coding for a particular antibody of interest have been isolated from such transgenic animals the genes coding for the constant regions can be replaced with human constant region genes in order to obtain a fully human antibody. The term “antibody” or “antibodies” as used herein, also refers to an aglycosylated antibody.

The term “fragment thereof” or grammatical variations thereof as used herein refers to an antibody fragment. A fragment of an antibody comprises at least one heavy or light chain immunoglobulin domain as known in the art and binds to one or more antigen(s). Examples of antibody fragments according to the invention include Fab, Fab′, F(ab′)2, and Fv and scFv fragments; as well as diabodies, triabodies, tetrabodies, minibodies, domain antibodies (dAbs), such as sdAbs, VHH or camelid antibodies (e.g. from camels or llamas such as Nanobodies™) and VNAR fragments, single-chain antibodies, bispecific, trispecific, tetraspecific or multispecific antibodies formed from antibody fragments or antibodies, including but not limited to Fab-Fv or Fab-Fv-Fv constructs. Antibody fragments as defined above are known in the art. If desired, an antibody or antigen binding fragment may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, antigen binding agents, synthetic (including PEG) or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

The effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents, which may be present on the above-mentioned synthetic polymers, include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

In one embodiment the polymer is albumin or a fragment thereof, such as human serum albumin or a fragment thereof. In one embodiment the polymer is a PEG molecule.

The size of the natural or synthetic polymer may be varied as desired, but will generally be in an average molecular weight range from 500 Da to 50000 Da, for example from 5000 to 40000 Da such as from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

Suitably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above.

The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment, Fab′ fragment or diFab which is PEGylated, i.e. has PEG (poly(ethyleneglycol)) covalently attached thereto, e.g. according to the method disclosed in EP 0948544 or EP1090037 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

Particular PEG molecules include 2-[3-(N-maleimido)propionamido]ethyl amide of N,N′-bis(methoxypoly(ethylene glycol) MW 20,000) modified lysine, also known as PEG2MAL40K (obtainable from Nektar, formerly Shearwater).

Alternative sources of PEG linkers include NOF who supply GL2-400MA3 (wherein m in the structure below is 5) and GL2-400MA (where m is 2) and n is approximately 450:

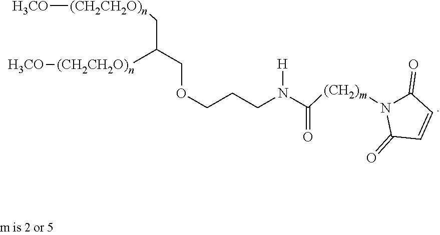

That is to say each PEG is about 20,000 Da.

Thus in one embodiment the PEG is 2,3-Bis(methylpolyoxyethylene-oxy)-1-{[3-(6-maleimido-1-oxohexyl)amino]propyloxy} hexane (the 2 arm branched PEG, —CH2) 3NHCO(CH2)5-MAL, Mw 40,000 known as SUNBRIGHT GL2-400MA3.

Further alternative PEG effector molecules of the following type:

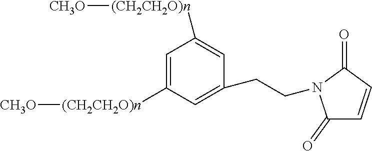

are available from Dr Reddy, NOF and Jenkem.

In one embodiment there is provided an antibody of the invention which is PEGylated (for example with a PEG described herein), attached through a cysteine amino acid residue at or about amino acid 226 in the chain, for example amino acid 226 of the heavy chain (by sequential numbering).

In one embodiment the present disclosure provides a Fab′PEG molecule comprising one or more PEG polymers, for example 1 or 2 polymers such as a 40 kDa polymer or polymers. Fab′-PEG molecules according to the present disclosure may be particularly advantageous in that they have a half-life independent of the Fc fragment. In one example the present invention provides a method for reducing the reconstitution time of a spray-dried protein formulation, wherein the method comprises spray-drying a protein formulation comprising a protein in the presence of a sugar and one or more amino acids, wherein the protein is a Fab′ fragment conjugated to a polymer such as PEG. In another embodiment of the present invention, there is provided a process for reducing the reconstitution time of a spray-dried protein formulation comprising the steps of:
a. Preparing a protein formulation comprising a protein, a sugar and one or more amino acids;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of a sugar and one or more amino acids and wherein the protein is Fab′ fragment conjugated to a polymer such as PEG, wherein the sugar is a disaccharide and is present in an amount from 1.0 to 20% w/v and wherein the one or more amino acids is present in an amount from or from above 50 mM to 200 mM.

The antibody or fragment thereof comprised in the protein formulation according to the methods, uses and processes of the present invention is preferably a human or humanized monoclonal antibody, preferably a humanized monoclonal full-length antibody.

Antibody molecules may be typically produced by culturing a host cell containing a vector encoding the antibody sequence under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

An antibody or an antigen-binding fragment thereof that can be manufactured according to industrial scales can be produced by culturing eukaryotic host cells transfected with one or more expression vectors encoding the recombinant antibody fragment. The eukaryotic host cells are preferably mammalian cells, more preferably Chinese Hamster Ovary (CHO) cells. Mammalian cells may be cultured in any medium that will support their growth and expression of the recombinant protein, preferably the medium is a chemically defined medium that is free of animal-derived products such as animal serum and peptone. There are different cell culture mediums available to the person skilled in the art comprising different combinations of vitamins, amino acids, hormones, growth factors, ions, buffers, nucleosides, glucose or an equivalent energy source, present at appropriate concentrations to enable cell growth and protein production. Additional cell culture media components may be included in the cell culture medium at appropriate concentrations at different times during a cell culture cycle that would be known to those skilled in the art.

Mammalian cell culture can take place in any suitable container such as a shake flask or a bioreactor, which may or may not be operated in a fed-batch mode depending on the scale of production required. These bioreactors may be either stirred-tank or air-lift reactors. Various large scale bioreactors are available with a capacity of more than 1,000 L to 50,000 L, preferably between 5,000 L and 20,000 L, or to 10,000 L. Alternatively, bioreactors of a smaller scale such as between 2 L and 100 L may also be used to manufacture an antibody or antibody fragment.

An antibody or antigen-binding fragment thereof is typically found in the supernatant of a mammalian host cell culture, typically a CHO cell culture. For CHO culture processes, wherein the protein of interest such as an antibody or antigen-binding fragment thereof is secreted in the supernatant, said supernatant is collected by methods known in the art, typically by centrifugation.

Alternatively, host cells are prokaryotic cells, preferably gram-negative bacteria. More preferably, the host cells are E. coli cells. Prokaryotic host cells for protein expression are well known in the art (Terpe, K. Appl Microbiol Biotechnol 72, 211-222 (2006)). The host cells are recombinant cells which have been genetically engineered to produce the protein of interest such as an antigen-binding fragment of an antibody. The recombinant E. coli host cells may be derived from any suitable E. coli strain including from MC4100, TG1, TG2, DHB4, DH5a, DH1, BL21, K12, XL1Blue and JM109. One example is E. coli strain W3110 (ATCC 27,325) a commonly used host strain for recombinant protein fermentations. Antibody fragments can also be produced by culturing modified E. coli strains, for example metabolic mutants or protease deficient E. coli strains.

An antibody fragment is typically found in either the periplasm of the E. coli host cell or in the host cell culture supernatant, depending on the nature of the protein, the scale of production and the E. coli strain used. The methods for targeting proteins to these compartments are well known in the art (Makrides, S. C.; Microbiol Rev 60, 512-538 (1996)). Examples of suitable signal sequences to direct proteins to the periplasm of E. coli include the E. coli PhoA, OmpA, OmpT, LamB and OmpF signal sequences. Proteins may be targeted to the supernatant by relying on the natural secretory pathways or by the induction of limited leakage of the outer membrane to cause protein secretion examples of which are the use of the pelB leader, the protein A leader, the co-expression of bacteriocin release protein, the mitomycin-induced bacteriocin release protein along with the addition of glycine to the culture medium and the co-expression of the kil gene for membrane permeabilization. Most preferably, the recombinant protein is expressed in the periplasm of the host E. coli.

Expression of the recombinant protein in the E. coli host cells may also be under the control of an inducible system, whereby the expression of the recombinant antibody in E. coli is under the control of an inducible promoter. Many inducible promoters suitable for use in E. coli are well known in the art and depending on the promoter expression of the recombinant protein can be induced by varying factors such as temperature or the concentration of a particular substance in the growth medium. Examples of inducible promoters include the E. coli lac, tac, and trc promoters which are inducible with lactose or the non-hydrolysable lactose analog, isopropyl-b-D-1-thiogalactopyranoside (IPTG) and the phoA, trp and araBAD promoters which are induced by phosphate, tryptophan and L-arabinose respectively. Expression may be induced by, for example, the addition of an inducer or a change in temperature where induction is temperature dependent. Where induction of recombinant protein expression is achieved by the addition of an inducer to the culture the inducer may be added by any suitable method depending on the fermentation system and the inducer, for example, by single or multiple shot additions or by a gradual addition of inducer through a feed. It will be appreciated that there may be a delay between the addition of the inducer and the actual induction of protein expression for example where the inducer is lactose there may be a delay before induction of protein expression occurs while any pre-existing carbon source is utilized before lactose. E. coli host cell cultures (fermentations) may be cultured in any medium that will support the growth of E. coli and expression of the recombinant protein. The medium may be any chemically defined medium such as e.g. described in Durany 0, et al. (2004) (Process Biochem 39, 1677-1684).

Culturing of the E. coli host cells can take place in any suitable container such as a shake flask or a fermenter depending on the scale of production required. Various large scale fermenters are available with a capacity of more than 1,000 liters up to about 100,000 liters. Preferably, fermenters of 1,000 to 50,000 liters are used, more preferably 1,000 to 25,000, 20,000, 15,000, 12,000 or 10,000 liters. Smaller scale fermenters may also be used with a capacity of between 0.5 and 1,000 liters.

Fermentation of E. coli may be performed in any suitable system, for example continuous, batch or fed-batch mode depending on the protein and the yields required. Batch mode may be used with shot additions of nutrients or inducers where required. Alternatively, a fed-batch culture may be used and the cultures grown in batch mode pre-induction at the maximum specific growth rate that can be sustained using the nutrients initially present in the fermenter and one or more nutrient feed regimes used to control the growth rate until fermentation is complete. Fed-batch mode may also be used pre-induction to control the metabolism of the E. coli host cells and to allow higher cell densities to be reached.

If desired, the host cells may be subject to collection from the fermentation medium, e.g. host cells may be collected from the sample by centrifugation, filtration or by concentration. In this case the process typically comprises a step of centrifugation and cell recovery prior to extracting the protein.

For E. coli fermentation processes wherein the protein of interest such as an antibody or antigen-binding fragment of an antibody is found in the periplasmic space of the host cell it is required to release the protein from the host cell. The release may be achieved by any suitable method such as cell lysis by mechanical or pressure treatment, freeze-thaw treatment, osmotic shock, extraction agents or heat treatment. Such extraction methods for protein release are well known in the art. Therefore, in a particular embodiment, the production process comprises an additional protein extraction step prior to protein purification.

Other methods for obtaining antigen-binding fragment of a human antibody in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody.

The method, use and process for reducing the reconstitution time of a spray-dried protein formulation.

The process of spray-drying involves evaporation of moisture after atomization of a fluid feed into fine droplets, resulting in a dried powder. Consisting of three basic steps, spray drying begins with dispersion of liquid feed in spray gas (compressed air or N2, 5-8 bar) by a two-fluid nozzle. Small droplets are sprayed through the nozzle (atomization). The droplets are then suspended in a drying medium, usually consisting of a heated co-current air stream, allowing evaporation and transfer of the liquid. In the final step, the dried solids are separated from the air stream in the cyclone. The dried powder is collected and the air is exhausted to the atmosphere.

Examples of spray-drying a protein formulation are described in the Examples hereinafter. The antibody or fragment thereof comprised in the protein formulation according to the present invention as a whole can be present at any concentration such as from 1 to 200 mg/mL, preferably from 20 to 150 mg/mL, even preferably from 30 to 100 mg/mL, such as 30, 35, 40, 45, 50, 55, 60, 65, 70, 75, 80, 85, 90, 95 or 100 mg/mL. Alternatively, the one or more amino acids is preferably present in the protein formulation to be spray-dried in an amount expressed in term of weight per 100 mL (% w/v). In such as case, the antibody or fragment thereof comprised in the protein formulation according to the present invention as a whole can be present in an amount of 0.1 to 20% w/v, preferably from 2.0 to 15% w/v, or even preferably from 3.0 to 10% w/v such as 3.0, 3.5, 4.0, 4.5, 5.0, 5.5, 6.0, 6.5, 7.0, 7.5, 8.0, 8.5, 9.0, 9.5 or 10% w/v.

The reduction of the reconstitution time of a protein formulation which has been spray-dried is achieved by incorporating prior spray-drying a sugar and one or more amino acids.

In the context of this invention as a whole, the sugar is preferably selected from a disaccharide, more preferably sucrose, trehalose or a mixture thereof. The sugar is preferably present in the protein formulation to be spray-dried in an amount from 1.0% to 20% w/v, or from 1.5% to 15% w/v or from 1.5% to 10% w/v or 1.5% to 4.5% w/v, such as 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.1, 4.2 or 4.5% w/v, or even preferably 2.5% to 4.2% w/v, such as 2.5, 2.6, 2.7, 2.8, 2.9, 3.0, 3.1, 3.2, 3.3, 3.4, 3.5, 3.6, 3.7, 3.8, 3.9, 4.0, 4.1 or 4.2% w/v. Alternatively, the sugar is preferably present in the protein formulation to be spray-dried in an amount expressed in molarity. In such as case, the sugar is preferably present in the protein formulation to be spray-dried in an amount from 30 to 600 mM, or from 45 to 135 mM, such as 45, 50, 55, 60, 65, 70, 75, 80, 85, 90, 95, 100, 110, 120 or 135 mM or even preferably 70 mM to 125 mM such as 70, 73, 75, 80, 85, 90, 95, 100, 105, 110, 115, 120 or 125 mM. Sucrose or trehalose are equally performing sugar for achieving the technical effect of the present invention and their individual presence over the mixture is preferred.

In the context of this invention as a whole, the one or more amino acids may be present in its D- and/or L-form, but the L-form is typical. The one or more amino acids is preferably glycine, L-proline, L-alanine, L-valine, L-serine, L-threonine, L-glutamine, L-asparagine, L-glutamate, L-aspartate, L-histidine, L-lysine, L-arginine or mixtures thereof. Although not limiting, the inclusion of a basic amino acid is preferred i.e. arginine, lysine and/or histidine or mixtures thereof. The amino acid may be present as any suitable salt e.g. a hydrochloride salt, such as arginine-HCl, Lysine-HCl or Histidine-HCl. The one or more amino acids is preferably present in the protein formulation to be spray-dried in an amount from or from above 10 mM to 250 mM, preferably from or from above 50 mM to 200 mM or from or from above 50 mM to 150 mM, such as 50, 51, 55, 60, 70, 80, 90, 100, 110, 120, 130, 140 or 150 mM. Alternatively, the one or more amino acids is preferably present in the protein formulation to be spray-dried in an amount expressed in term of weight per 100 mL (% w/v). For instance, should the at least one amino acid be Arginine-HCl (having a MW of 210.66 Da), said Arginine-HCl will be present in an amount of from or from above 0.2 to 5.25% w/v weight or from or from above 1.06 to 4.2% w/v, preferably from or from above 1.06 to 3.2% w/v, such as 1.1, 1.15, 1.2, 1.5, 2.0, 2.2, 2.5, 3.0 or 3.2% w/v. In another example, should the at least one amino acid be Lysine-HCl (having a MW of 182.65 Da), said lysine-HCl will be present in an amount of from or from above 0.1 to 4.5% w/v weight or from or from above 0.9 to 3.6% w/v, preferably from or from above 0.9 to 2.7% w/v, such as 0.9, 0.95, 1.0, 1.5, 1.8, 2.0, 2.5 or 2.7% w/v. It would be well within the skills of the skilled person to convert the molarity of interest into the % w/v for any amino acid.

In the context of this invention as a whole, the molarity of the sugar and the one of the one or more amino acids can be cumulated. In such a case, the sugar and the one or more amino acids are preferably present in the protein formulation to be spray-dried at a cumulative molarity (also alternatively referred to as cumulative amount) of from or from above 60 mM to 400 mM, preferably from or from above 70 mM to 300 mM or from or from above 70 mM to 260 mM, such as 70, 80, 90, 100, 110, 120, 130, 140, 150, 160, 170, 180, 190, 200, 210, 220, 230, 240, 250 or 260 mM.

In one embodiment, the method for reducing the reconstitution time of a spray-dried protein formulation according to the invention comprises spray-drying a protein formulation comprising a protein in the presence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. Said antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof, wherein the sugar is a disaccharide and is present in an amount from 1.0 to 20% w/v and wherein the one or more amino acids is present in an amount from or from above 50 mM to 200 mM. Preferably, the protein is an antibody or a fragment thereof. Said antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, from 1% to 20% of sucrose or trehalose or a mixture thereof and from or from above 50 mM to 250 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. Said antibody or a fragment thereof is optionally conjugated to a polymer such as PEG.

In yet another embodiment, the method for reducing the reconstitution time of a spray-dried protein formulation according to the invention comprises spray-drying a protein formulation comprising a protein in the presence of from 1.5% to 4.5% w/v or from 2.5% to 4.2% w/v of sucrose or trehalose or a mixture thereof and from or from above 50 mM to 200 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. Said antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, from 1.5% to 4.5% w/v or from 2.5% to 4.2% w/v of sucrose or trehalose or a mixture thereof and from or from above 50 mM to 200 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. Said antibody or a fragment thereof is optionally conjugated to a polymer such as PEG. The present invention also provides for a method for reducing the reconstitution time of a spray-dried protein formulation comprising spray-drying a protein formulation comprising a protein in the presence of a sugar, one or more amino acids and a surfactant.

In addition, the present invention provides for a process for reducing the reconstitution time of a spray-dried protein formulation according to the invention comprises the steps of:
a. Preparing a protein formulation comprising a protein, a sugar, one or more amino acids and a surfactant;
b. Spray-drying the protein formulation prepared in step a);
c. Recovering the spray-dried protein formulation of step b);
d. Reconstituting, preferably with water, the recovered spray-dried protein formulation within a reconstitution time RT1;

wherein the reconstitution time RT1 is less than the reconstitution time of the same protein formulation prepared in the absence of a sugar and one or more amino acids. Preferably, the protein is an antibody or a fragment thereof, wherein the sugar is a disaccharide and is present in an amount from 1.0 to 20% w/v and wherein the one or more amino acids is present in an amount from or from above 50 mM to 200 mM.

Surfactants available for use in the methods according to the invention include, but are not limited to, non-ionic surfactants, ionic surfactants and zwitterionic surfactants. Typical surfactants for use with the invention include, but are not limited to, sorbitan fatty acid esters (e.g. sorbitan monocaprylate, sorbitan monolaurate, sorbitan monopalmitate), sorbitan trioleate, glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils {e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide); C₁₀-C₁₈ alkyl sulfates (e.g. sodium cetyl sulfate, sodium lauryl sulfate, sodium oleyl sulfate), polyoxyethylene C₁₀-C₁₈ alkyl ether sulfate with an average of 2 to 4 moles of ethylene oxide units added (e.g. sodium polyoxyethylene lauryl sulfate), and C₁-C₁₈ alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C₁₂-C₁₈ fatty acids. Preferred surfactants are polyoxyethylene sorbitan fatty acid esters e.g. polysorbate 20, 40, 60 or 80, more preferably, polysorbate 20 or polysorbate 80.

Surfactant are generally present in an amount from 0.01% w/v to 1.0% w/v, preferably from 0.02% w/v to 0.1% w/v or even preferably from 0.02% w/v to 0.05% w/v. Preferably the surfactant is polysorbate 20 or polysorbate 80 and it is present in an amount from 0.02% w/v to 0.05% w/v, such as 0.02, 0.03, 0.04 or 0.05% w/v.

In one embodiment, the method for reducing the reconstitution time of a spray-dried protein formulation according to the invention comprises spray-drying a protein formulation comprising a protein in the presence of sucrose or trehalose or a mixture thereof, one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof and a surfactant selected from a polysorbate. Preferably, the protein is an antibody or a fragment thereof and/or the polysorbate is polysorbate 20. The antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, sucrose or trehalose or a mixture thereof, one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof and a polysorbate surfactant;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof, wherein the sugar is a disaccharide and is present in an amount from 1.0 to 20% w/v and wherein the one or more amino acids is present in an amount from or from above 50 mM to 200 mM. Preferably, the protein is an antibody or a fragment thereof and/or the polysorbate surfactant is polysorbate 20. The antibody or a fragment thereof is optionally conjugated to a polymer such as PEG.

In another embodiment, the method for reducing the reconstitution time of a spray-dried protein formulation according to the invention comprises spray-drying a protein formulation comprising a protein in the presence of from 1% to 20% w/v of sucrose or trehalose or a mixture thereof, from 50 mM to 250 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof and from 0.01% w/v to 1.0% w/v of polysorbate 20. Preferably, the protein is an antibody or a fragment thereof. The antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, from 1% to 20% of sucrose or trehalose or a mixture thereof, from 10 mM to 250 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof and from 0.01% w/v to 1.0% w/v of polysorbate 20; b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose or a mixture thereof and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. The antibody or a fragment thereof is optionally conjugated to a polymer such as PEG.

In yet another embodiment, the method for reducing the reconstitution time of a spray-dried protein formulation according to the invention comprises spray-drying a protein formulation comprising a protein in the presence of from 1.5% to 4.5% w/v or from 2.5% to 4.2% w/v of sucrose or trehalose or a mixture thereof, from or from above 50 mM to 200 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof and from 0.02% w/v to 0.1% w/v of polysorbate 20. The antibody or a fragment thereof is optionally conjugated to a polymer such as PEG.

In another embodiment, the process for reducing the reconstitution time of a spray-dried protein formulation according to the invention comprises the steps of:
a. Preparing a protein formulation comprising a protein, from 1.5% to 4.5% w/v or from 2.5% to 4.2% w/v of sucrose or trehalose or a mixture thereof, from or from above 50 mM to 200 mM of one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof and from 0.02% w/v to 0.1% w/v of polysorbate 20;
b. Spray-drying the protein formulation prepared in step a);
c. Recovering the spray-dried protein formulation of step b);
d. Reconstituting, preferably with water, the recovered spray-dried protein formulation within a reconstitution time RT1;
   wherein the reconstitution time RT1 is less than the reconstitution time of the same protein formulation prepared in the absence of sucrose or trehalose and one or more amino acids selected from the group of glycine, L-proline, L-alanine, L-valine, L-serine, L-threonine, L-glutamine, L-asparagine, L-glutamate, L-aspartate, L-histidine, L-lysine, L-arginine or mixtures thereof. Preferably, the protein is an antibody or a fragment thereof. The antibody or a fragment thereof is optionally conjugated to a polymer such as PEG.

The protein formulation to be spray-dried has a pH of from 4.0 to 7.5, preferably of from 4.0 to 6.0, more preferably of from 5.0 to 6.0, such as 5.0, 5.1, 5.2, 5.3, 5.4, 5.5, 5.6, 5.7, 5.8, 5.9 or 6.0. To maintain the pH constant, the protein formulation to be spray-dried comprises a buffering agent. There are many buffering agents used in the pharmaceutical field of protein formulations, such as, but not limited to, citrate, phosphate, lactate, histidine, glutamate, maleate, tartrate, or succinate. A preferred buffer species is typically selected amongst those having a pKa that is close (+/−1 pH unit) to the preferred pH for optimal protein stability in order to maintain high buffering capacity, and is associated with the maximal demonstrated stability observed for a particular protein when placed in a series of varied buffer species. The adequate pH ranges of a formulation are generally chosen from those associated with the maximal demonstrated stability observed for a particular protein when placed in a series of varied pH formulations. The buffering agent may also be an amino acid or a mixture of amino acids, preferably at a concentration of 10 mM to 100 mM, 10 mM to 80 mM, 10 mM to 60 mM, 15 mM to 60 mM, preferably 10 mM to 50 mM, such as 10, 15, 20, 25, 30, 35, 40, 45 or 50 mM. As a none limiting example, the buffer can be a histidine buffer at a concentration of 10 mM to 100 mM.

Additional excipients may be used in the methods and processes according to the present invention. These excipients include, but are not limited to, viscosity enhancing agents, bulking agents, solubilising agents such as monosaccharides, e.g., fructose, maltose, galactose, glucose, D-mannose, sorbose and the like; polysaccharides, e.g. raffinose, melezitose, maltodextrins, dextrans, starches, and the like; and polyols, such as mannitol, xylitol, maltitol, lactitol, xylitol sorbitol (glucitol) and the like; poly-ethylene glycols (e.g. PEG100, PEG300, PEG600, PEG1500, PEG2000, PEG3000, PEG3350, PEG4000, PEG6000, PEG8000 or PEG20000), polyvinylpyrrolidone, trimethylamine N-oxide, trimethylglycine or combinations thereof.

Before a spray-dried protein formulation composition can be administered to a patient it needs to be reconstituted with a solvent. Within the present invention, the preferred solvent is an aqueous solvent, more preferably the aqueous solvent is water, even more preferably is sterilized water.

It will be understood by one skilled in the art that the reconstitution of the spray-dried protein formulation can be performed days, weeks or months after said spray-dried protein formulation according to the invention have been recovered.

The volume of solvent used for reconstitution dictates the concentration of the protein, such as an antibody or fragment thereof, in the resulting reconstituted spray-dried protein formulation. Reconstitution with a smaller volume of solvent than the pre-spray-drying volume provides a formulation which is more concentrated than before spray-drying and vice-versa. The reconstitution ratio (volume of pre-spray-dried protein formulation to solvent used to reconstitute the spray-dried protein formulation) may vary from 1:0.1 to 10:1. In a preferred embodiment, a ratio of about 1:0.5 is applied so that the resulting concentration of the protein in the reconstituted spray-dried protein formulation is twice the concentration of the protein formulation before spray-drying.

The present invention also provides for a spray-dried protein formulation obtained through the process according to the present invention. Preferably such a spray-dried protein formulation is an antibody formulation or a fragment of an antibody formulation.

Such spray-dried protein formulation may be stored, until reconstitution is required, into a suitable container such as a vial, an ampoule, a tube, a bottle or a syringe (such as a pre-filled syringe). The container may be part of a kit-of-parts comprising one or more containers comprising the spray-dried protein formulation obtained according to the process of the present invention, suitable solvents for reconstituting the spray-dried protein formulation, delivery devices such as a syringe, pre-filled syringe, an autoinjector, a needleless device, an implant or a patch, or other devices for parental administration and instructions of use.

The protein formulation or the spray-dried protein formulation (alternatively named reconstituted protein formulation once reconstituted), obtained through the processes according to the present invention is for use in therapy or diagnosis.

The reconstituted protein formulations obtained through the processes according to the invention are administered in a therapeutically effective amount. The term “therapeutically effective amount” as used herein refers to an amount of a protein (i.e. an antibody) needed to treat, ameliorate or prevent a targeted disease, disorder or condition, or to exhibit a detectable therapeutic, pharmacological or preventative effect. For any antibody or antigen-binding fragments thereof, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount of antibody will be from 0.01 mg/kg to 500 mg/kg, for example 0.1 mg/kg to 200 mg/kg or 1 mg/kg to 100 mg/kg.

The appropriate dosage will vary depending upon, for example, the particular antibody to be employed, the subject treated, the mode of administration and the nature and severity of the condition being treated. In a particular embodiment, the reconstituted spray-dried protein formulation obtained through the processes according to the present invention is administered by subcutaneous route or as an intramuscular injection.

The invention will now be further described by way of examples with references to embodiments illustrated in the accompanying drawings.

### EXAMPLES

### Example 1

A humanized IgG monoclonal antibody (mAb1), having an isoelectric point (pl) of about 6.1, was provided in an aqueous solution at a concentration of 160 mg/ml in 30 mM histidine, 200 mM sorbitol pH5.6. All excipients were obtained from Sigma-Aldrich (Steinheim™, Germany). Amicon Ultra-15 30K (Millipore™) centrifugal filters for volumes up to 15 ml were used to perform a buffer exchange. Four cycles of 2-3 times dilution were performed, including repeatedly centrifugation for 2 hours at 20° C. (relative centrifugal force=4000·g). Stocks solutions were prepared in 15 mM histidine buffer at pH 5.6. All formulation comprised 2.5% sucrose (equivalent to 73 mM sucrose). The concentrations of the amino acids used in this study is shown in Table 1.

TABLE 1

| Amino acid | |
|---|---|
| (In addition to buffer and 2.5% sucrose) | mM |
| glycine | 100 |
| L-serine | 100 |
| L-cysteine | 100 |
| L-isoleucine | 75 |
| L-glutamine | 100 |
| L-alanine | 100 |
| L-arginine HCl | 75 |
| L-proline | 100 |
| L-leucine | 75 |
| L-phenylalanine | 75 |
| L-lysine HCl | 100 |
| L-tryptophan | 25 |

After buffer exchange, formulations were diluted with the corresponding stock solutions to a concentration of mAb1 of 50 mg/ml before spray-drying.

Spray drying was performed with a Büchi Mini Spray dryer B-290, with outlet air passing through a dehumidifier (Büchi Labortechnik, Flawil, Switzerland). Inlet temperature was set at 120° C. and outlet temperature was between 55 and 60° C. The aspirator was set at 100%, which corresponds to an air flow rate of 35 m3/h. Rate setting for the liquid feed flow was 3 ml/min, according to 10% pump rate, and 6001/h for the atomizing N2 flow. The formulations (9-15 ml) were atomized with a two-fluid nozzle (0.7 mm liquid orifice internal diameter). After spray drying, the powders were collected through a cyclone in a glass container, transferred in a plastic vial, and stored in the fridge at 2-8° C. For each formulation four vials were filled with spray dried powder, containing approximately 100 mg of mAb1. Three vials were used for reconstitution time evaluation.

Spray dried formulations were reconstituted with 0.9 ml MilliQ-water to a concentration of approximately 100 mg/ml. The time for the powder to completely dissolve, was visually observed and recorded. A triplicate of samples was reconstituted to investigate the variation in reconstitution time for a given formulation. Upon reconstitution, the concentration of mAb1 was determined undiluted by UV absorbance at 280 nm with a SoloVPE (CE Technologies) connected to a Cary50Bio UV-visible spectrophotometer (Varian), with extinction coefficient ε=1.33 ml·mg-1·cm−1.

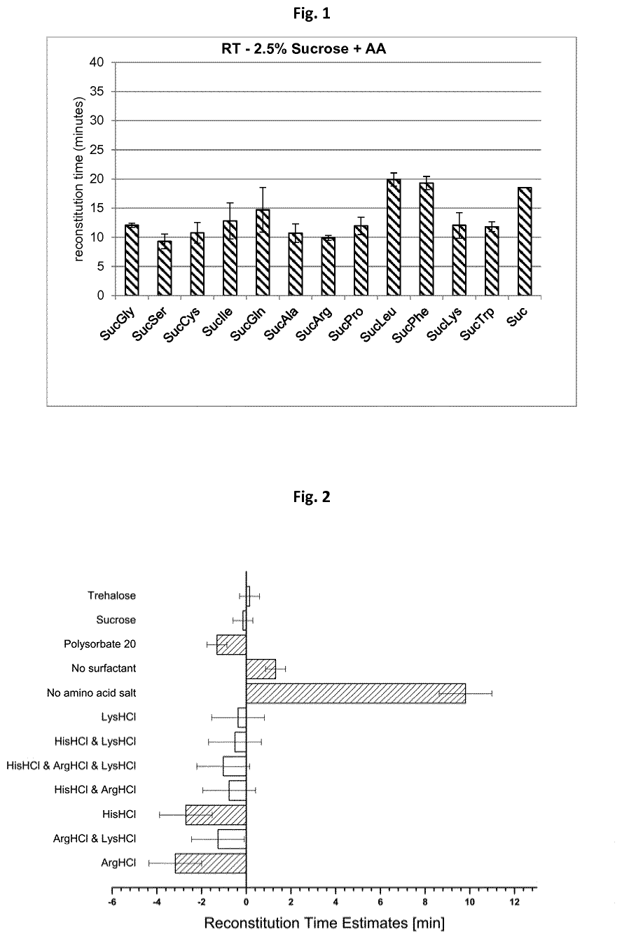

Spray-dried formulations were reconstituted in triplicate, mean and standard deviation were calculated for the different spray-dried mAb1 formulations. All formulations showed faster reconstitution time compared to 2.5% sucrose, except for formulations with leucine and phenylalanine (FIG. 1). The formulation comprising leucine showed a very milky appearance and presence of a high amount of aggregates (data not shown) which affected the reconstitution time. The formulation comprising phenylalanine was instead clear and showed little formation of (sub)visible particles (data not shown), but still resulted in a higher than sucrose alone reconstitution time. These experiments show that the combination of a sugar, such as sucrose, in combination with an amino acid, such as glycine, serine, cysteine, isoleucine, glutamine, alanine, arginine, proline, lysine and tryptophan reduces the reconstitution time of a spray-dried antibody formulation in comparison to sucrose alone.

### Example 2

The effect of a surfactant on the reduction of reconstitution time was analysed. Tangential flow diafiltration was used to exchange the mAb1 storage buffer with an aqueous stock solution (pH 5.0) containing either a sugar (0.1 M) and lactate (0.05 M), or L-arginine HCl (0.24 M), trehalose (0.1 M) and L-histidine (0.05 M). After the diafiltration step, the solutions were further concentrated and filtered (Polyethersulfone, 0.22 um, Merck Millipore, Bedford Mass., U.S.). The concentration of the mAb1 formulations was measured using UV absorbance at 280 nm with extinction coefficient 1.33 (mg/ml)⁻¹ cm⁻¹, and adjusted to 100 mg/ml. Feed solutions used during the formulation screening were prepared by adding filtered (0.22 um) solutions of the remaining excipients. Solutions used during the SD process parameter screening experiments (Table 2), were spiked with polysorbate 20 and diluted with ultra-pure water (Type 1 (ρ≥18.2 MΩcm at 25° C.) and filtered (0.22 um)) to their final concentrations. The sodium hydroxide stock solution, L-histidine monohydrochloride monohydrate and polysorbate 20 were obtained from Merck (Darmstadt, Germany). L-arginine monohydrochloride, L-lysine monohydrochloride and D(+)-trehalose dihydrate were obtained from Sigma-Aldrich (St. Louis, Mo., U.S.). D(+)-Sucrose was purchased from Applichem (Darmstadt, Germany). L-histidine was purchased from Merck and Sigma-Aldrich for the formulation and spray drying (SD) process parameter screening formulations, respectively. Lactic acid and hydrochloric acid stock solutions were provided by Fisher scientific (Pittsburgh, Pa., U.S.).

Feed solutions for the formulation screening experiments were spray dried at a concentration of mAb1 of 50 mg/ml and a total feed volume of 15 ml, using a Büchi B-290 Mini Spray Dryer, equipped with a 0.7 mm two-fluid nozzle, high performance cyclone, small collection vessel and the B-296 Dehumidifier (Büchi Labortechnik AG, Flawil, Switzerland). Settings were based on in-house procedure and kept constant for all formulation screening experiments. Inlet air temperature was set at 120° C. (outlet temperature was monitored and ranged between 55-60° C.), inlet air flow rate at 580 l/min, nozzle N² flow rate at 10 l/min and the solution feed rate was set at 3 ml/min.

The powder from the collection vessel (collector) was then pooled with the powder recovered from the cyclone and dispensed into 2 ml Type I, clear, tubular glass injection vials (Schott AG, Mainz, Germany) closed with FluroTec rubber injection stoppers (West pharmaceutical services, West Whiteland Township, Pennsylvania, U.S.) and aluminium crimp seals (Adelphi healthcare packaging, West Sussex, U.K.). Finally, samples were stored either at 5° C. or at 40° C. during 4 weeks, followed by storage at 5° C. prior to reconstitution.

TABLE 2

| | | | | Sugar | Surfactant |
|---|---|---|---|---|---|
| | Annino Acid Salts | mM | | (50 mM; ≈1.7%) | (2 mM) |
| 1 | No amino acid salt | 0 | | Sucrose | No surfactant |
| 2 | HisHCl | 120 | mM | Sucrose | No surfactant |
| 3 | ArgHCl | 120 | mM | Sucrose | No surfactant |
| 4 | LysHCl | 120 | mM | Sucrose | No surfactant |
| 5 | HisHCl & ArgHCl | 60 | mM each | Sucrose | No surfactant |
| 6 | HisHCl & LysHCl | 60 | mM each | Sucrose | No surfactant |
| 7 | ArgHCl & LysHCl | 60 | mM each | Sucrose | No surfactant |
| 8 | HisHCl & ArgHCl & LysHCl | 40 | mM each | Sucrose | No surfactant |
| 9 | No amino acid salt | 0 | | Trehalose | No surfactant |
| 10 | HisHCl | 120 | mM | Trehalose | No surfactant |
| 11 | ArgHCl | 120 | mM | Trehalose | No surfactant |
| 12 | LysHCl | 120 | mM | Trehalose | No surfactant |
| 13 | HisHCl & ArgHCl | 60 | mM each | Trehalose | No surfactant |
| 14 | HisHCl & LysHCl | 60 | mM each | Trehalose | No surfactant |
| 15 | ArgHCl & LysHCl | 60 | mM each | Trehalose | No surfactant |
| 16 | HisHCl & ArgHCl & LysHCl | 40 | mM each | Trehalose | No surfactant |
| 17 | No amino acid salt | 0 | | Sucrose | Polysorbate 20 |
| 18 | HisHCl | 120 | mM | Sucrose | Polysorbate 20 |
| 19 | ArgHCl | 120 | mM | Sucrose | Polysorbate 20 |
| 20 | LysHCl | 120 | mM | Sucrose | Polysorbate 20 |
| 21 | HisHCl & ArgHCl | 60 | mM each | Sucrose | Polysorbate 20 |
| 22 | HisHCl & LysHCl | 60 | mM each | Sucrose | Polysorbate 20 |
| 23 | ArgHCl & LysHCl | 60 | mM each | Sucrose | Polysorbate 20 |
| 24 | HisHCl & ArgHCl & LysHCl | 40 | mM each | Sucrose | Polysorbate 20 |
| 25 | No amino acid salt | 0 | | Trehalose | Polysorbate 20 |
| 26 | HisHCl | 120 | mM | Trehalose | Polysorbate 20 |
| 27 | ArgHCl | 120 | mM | Trehalose | Polysorbate 20 |
| 28 | LysHCl | 120 | mM | Trehalose | Polysorbate 20 |
| 29 | HisHCl & ArgHCl | 60 | mM each | Trehalose | Polysorbate 20 |
| 30 | HisHCl & LysHCl | 60 | mM each | Trehalose | Polysorbate 20 |
| 31 | ArgHCl & LysHCl | 60 | mM each | Trehalose | Polysorbate 20 |
| 32 | HisHCl & ArgHCl & LysHCl | 40 | mM each | Trehalose | Polysorbate 20 |

Data obtained for the different factor levels of the formulation screening design were used to fit regression models, containing nominal factors, for each of the responses. As the JMP software represents nominal variables by terms whose parameter estimates average to zero across all the levels, n-level nominal factors will be represented by n−1 indicator variables for processing. Therefore only n−1 parameter estimates could be obtained directly for the formulation screening since the parameter estimates were calculated by taking the difference between the average response corresponding to a certain level and the average response across all levels. Parameter estimates for the final factor level were then calculated separately based on the knowledge that parameter estimates across all levels of a nominal variable are constrained to sum to zero, i.e. the final term was calculated as the negative of the sum of the estimates across the other n−1 levels. This implies a dependency between the expanded parameter estimates (JMP Genomics 8 manual).

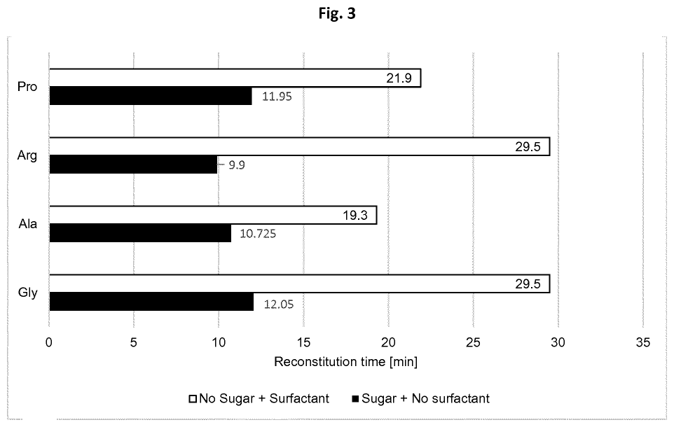

The spray-dried formulations were reconstituted in triplicate at 100 mg/ml, twice the mAb1 pre-spray-drying concentration in the feed solutions, and the expanded parameter estimates for the resulting model are shown in FIG. 2. Parameter estimates were significant at the 0.05 level for the surfactant factor and the single amino acid salt levels of L-arginine HCl and L-histidine HCl.

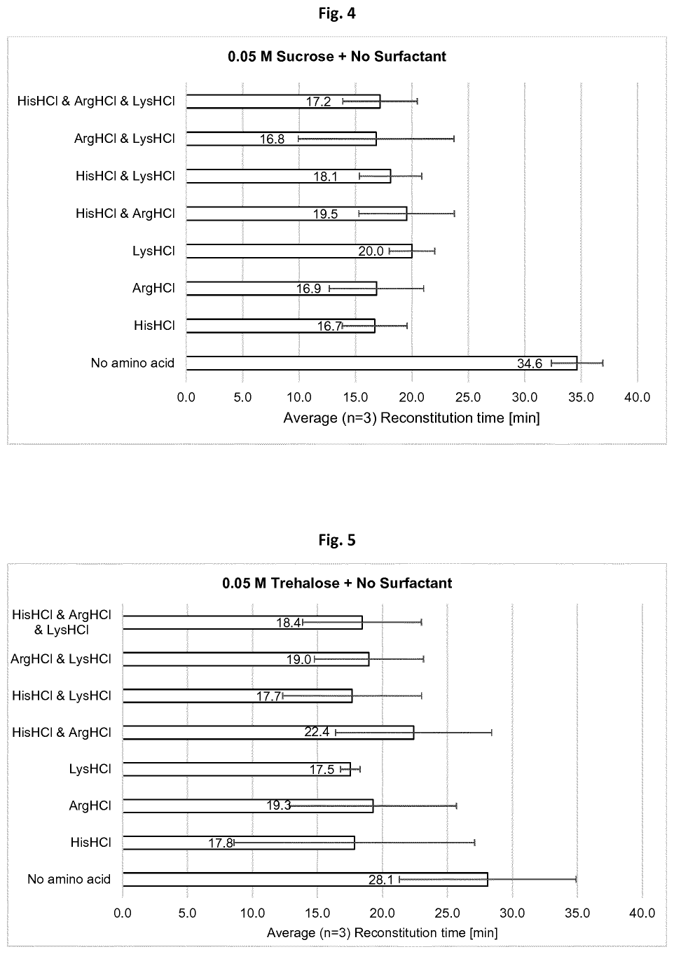

The addition of polysorbate 20 instead of a sugar did not result in a reduction of the reconstitution time. FIG. 3 shows a comparison of the reconstitution times obtained for spray-dried formulations of mAb1 comprising sucrose and glycine, sucrose and alanine, sucrose and arginine and sucrose and proline of Example 1 versus spray-dried formulations comprising polysorbate 20 instead of sucrose. As shown in FIG. 3, the reconstitution times were in each case worse for the amino acids in combination with the surfactant in the absence of sucrose.

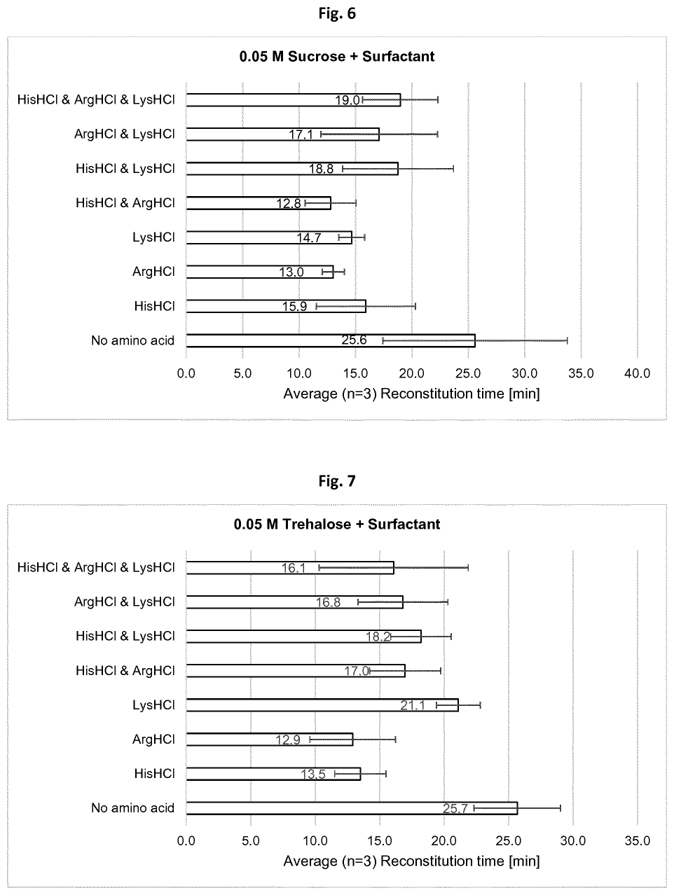

Similarly, as shown in FIGS. 4, 5, 6 and 7 the reconstitution time was not reduced for a formulation spray-dried in the presence of a surfactant (polysorbate 20) and a sugar (sucrose as shown in FIGS. 4 and 5 or trehalose as shown in FIGS. 6 and 7) in the absence of one or more amino acids such as L-arginine HCl, L-lysine HCl, L-histidine HCL or combinations thereof.

### Example 3

A formulation comprising 200 mg/ml of a Fab′-PEG antibody, having a pl of about 7.0, was buffer exchanged using Amicon Ulta-15-30K (Millipore) centrifugal filter, with various formulations of amino acids (glycine, alanine, proline, lysine, serine, glutamine and arginine) and sucrose in 10 mM histidine buffer pH 5.5.

The formulation prepared were as shown in Table 3.

TABLE 3

| Amino acid | | |
|---|---|---|
| (in addition to 2.5% sucrose = 73 mM) | (%) | mM |
| Glycine | 0.25-2.5 | 34-333 |
| Lysine | 1 | 68 |
| Proline | 1 | 87 |
| Alanine | 1 | 112 |
| Serine | 1 | 95 |
| Glutamine | 1 | 68 |
| Arginine | 1 | 57 |

Spray-drying of the formulations was performed with a Büchi B290 mini spray-dryer, coupled to a dehumidifier B-296 used to pre dehumidify the drying air before spray-drying (no settings required). A formulation comprising 50 mg/mL of a Fab′-PEG antibody was spray-dried using the process parameters shown in Table 4.

TABLE 4

| Parameter | Value | |
|---|---|---|
| Aspiration rate (Air flow | 35 m³/h (setting: 100%) | |
| inlet) | | |
| Air Tinlet | 120° | C. |
| Nozzle air flow | 600 L/h N2 (setting: maximum) | |
| Nozzle | 0.7 | mm |
| Pump rate | ~145 mL/h (setting 10%) | |
| Air Toutlet measured | 53-72° | C. |

Spray-dried products were reconstituted with 900 μL of MilliQ water to obtain a concentration of 100 mg/mL. The time for the powder to completely dissolve to a clear solution was measured. At t0 triplicate of samples were reconstituted to evaluate the variation in reconstitution time for a given formulation. The Fab′-PEG concentration was determined by UV absorption at 280 nm undiluted with SoioVPE (CE Technologies) connected to a Cary50Bio UV-Visible spectrophotometer (Varian) or after dilution to approximately 1 mg/mL with a Spectramax M5 plate reader (Molecular Devices), with c=0.86 mL·mg−1·cm−1.

As shown in FIG. 8, the reconstitution time of a spray-dried formulation of a Fab′-PEG antibody at 100 mg/ml in the presence of 2.5% sucrose and increasing concentration of glycine is reduced in comparison to the same formulation with no glycine (around 25 minutes).

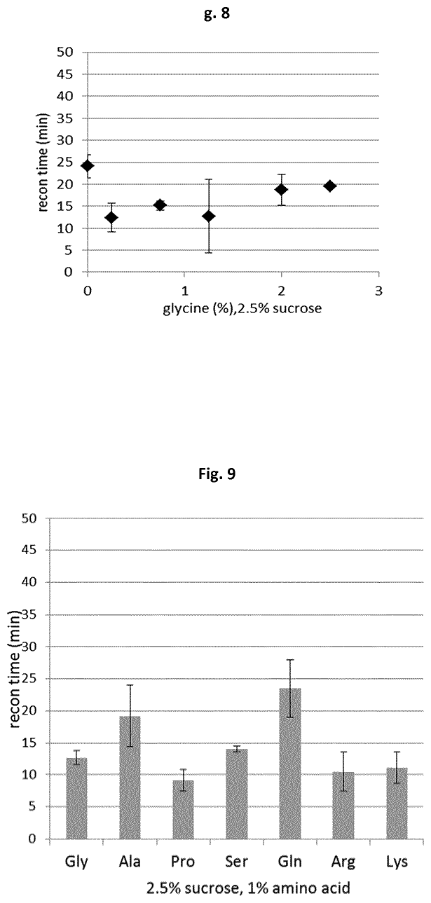

The other amino acids tested also showed superior effect with respect to the reconstitution time of a formulation comprising a Fab′-PEG antibody in 2.5% sucrose only. As shown in FIG. 9, the inclusion of the selected amino acids lead to a reduction in reconstitution times with respect to sucrose alone (Gln>Ala>Ser>Gly>Lys>Arg>Pro).

### Example 4

The effect of addition of amino acids and/or sugar on the reconstitution time was further analysed. mAb1 was provided in an aqueous solution at a concentration of 50 mg/ml in 15 mM histidine, pH 5.6. Different amino acids (Arg-HCl; Gly-HCl, Lys-HCl and Pro-HCl) and one sugar (trehalose) were each tested at different molarities (0, 50, 100 and 150 mM for the amino acids and 30, 75, 120 mM for the sugar).

The formulation prepared were as shown in Table 5.

TABLE 5

| Amino acid | Sugar |
|---|---|
| (either Arg; Gly, Lys or Pro; in mM) | (Treahalose; mM) |
| 0 | 30 |
| 0 | 75 |
| 0 | 120 |
| 50 | 30 |
| 50 | 75 |
| 50 | 120 |
| 100 | 30 |
| 100 | 75 |
| 100 | 120 |
| 150 | 30 |
| 150 | 75 |
| 150 | 120 |

Spray-drying of the formulations, reconstitution and assessment of reconstitution time were performed as per example 3.

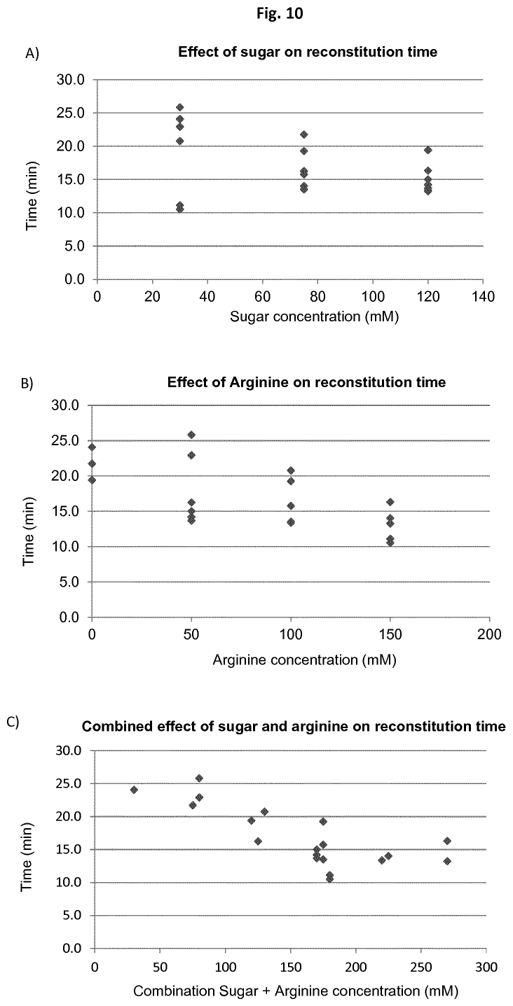

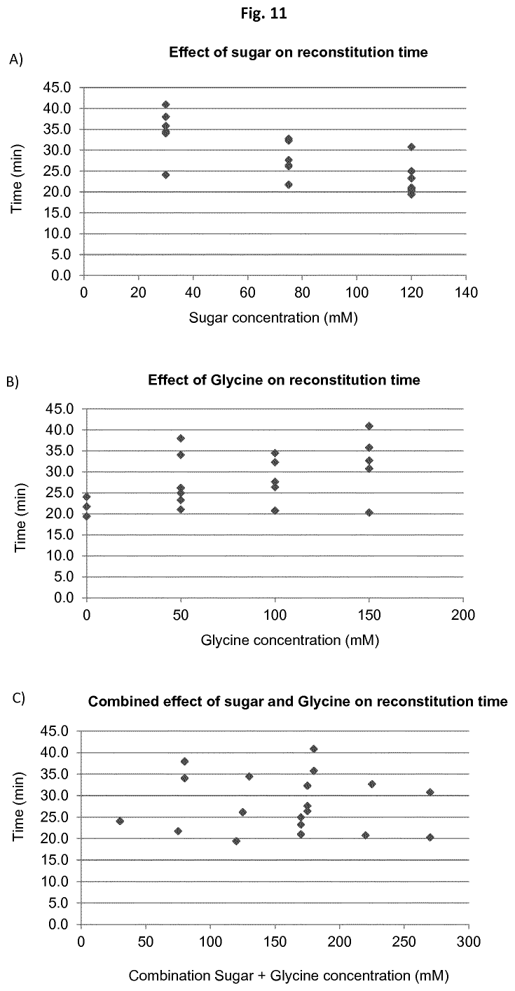

As shown in FIG. 10B, the reconstitution time of a spray-dried formulation of mAb1 at 50 mg/ml (100 mg/mL upon reconstitution) in the presence of a sugar (herein the disaccharide trehalose, whatever its molarity) and increasing concentration of Arginine is reduced in comparison to the same formulation with no arginine (around 22 minutes). FIG. 10C underlines that this effect is further increased when both the molarity of sugar and amino acid are combined (cumulative molarity), until reaching a plateau (herein around 200 mM cumulative molarity).

In comparison, the presence of glycine does not improve the reconstitution time for mAb1, whatever its molarity or the one of sugar.

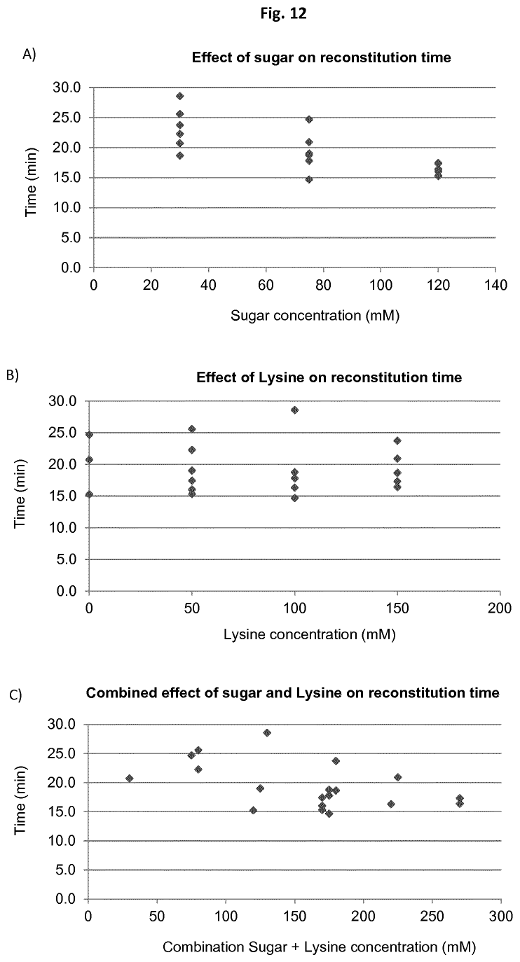

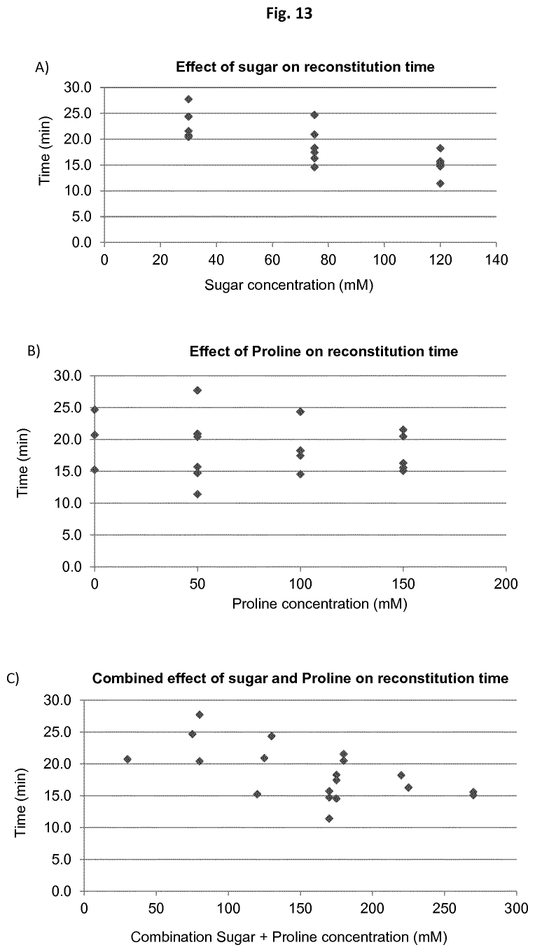

The other tested amino acids (Proline and Lysine) also showed a trend for reduced reconstitution time when considering cumulative molarity (see FIGS. 12C and 13C), although less pronounced than with Arginine.

### Example 5

The effect of addition of amino acids and sugar on the reconstitution time was further analysed with mAb2 (a humanized IgG monoclonal antibody, having a pl of about 7.6), at 50 mg/mL (100 mg/mL upon reconstitution). Different amino acids (Arg-HCl; Gly-HCl, Lys-HCl and Pro-HCl) and one sugar (trehalose) were tested at only one molarity (100 mM for the amino acids and 75 mM for the sugar), in a 15 mM Histidine buffer, pH 5.6, in presence of 0.01% w/v PS20 (0.02% w/v upon reconstitution). The control formulation contained no amino acid. Spray-drying of the formulations, reconstitution and assessment of reconstitution time were performed as per example 3.

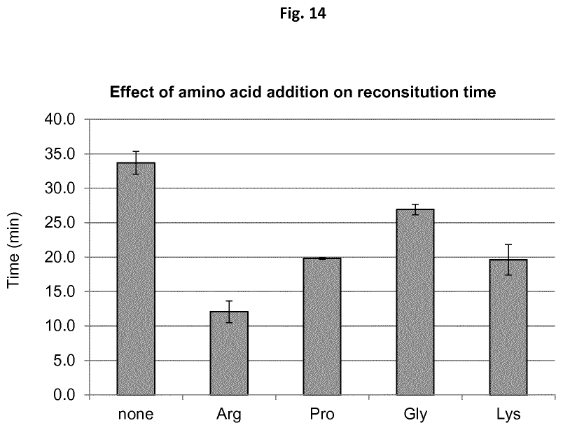

FIG. 14 underlines that although all of the amino acids tested are able to reduce the reconstitution time compared to a formulation comprising only 75 mM sugar, the best results are obtained with Arginine, followed by Proline and Lysine.

1. A method for reducing reconstitution time of a spray-dried protein formulation, wherein the method comprises spray-drying a protein formulation comprising a protein in the presence of a sugar and one or more amino acids, wherein the sugar is a disaccharide and is present in an amount from 1.0 to 20% w/v and wherein the one or more amino acids is present in an amount from or from above 50 mM to 200 mM.

2. The method according to claim 1, wherein the protein is an antibody or a fragment thereof.

3. The method according to claim 1, wherein the sugar is sucrose, trehalose or a mixture thereof.

4. The method according to claim 1, wherein the one or more amino acids is glycine, L-proline, L-alanine, L-valine, L-serine, L-threonine, L-glutamine, L-asparagine, L-histidine, L-lysine, L-arginine or mixtures thereof.

5. The method according to claim 1, wherein the sugar is sucrose or trehalose and the one or more amino acid is L-arginine hydrochloride, L-histidine hydrochloride, L-lysine hydrochloride or mixtures thereof.

6. The method according to claim 1, wherein the method comprises spray-drying a protein formulation further comprising a surfactant.

7. The method according to claim 6, wherein the surfactant is a polysorbate.

8. A process for reducing reconstitution time of a spray-dried protein formulation comprising:
preparing a protein formulation comprising a protein, a sugar and one or more amino acids;
b. spray-drying the protein formulation prepared in step a);
c. recovering the spray-dried protein formulation of step b); and
d. reconstituting the recovered spray-dried protein formulation within a reconstitution time RT1;
wherein the reconstitution time RT1 is less than the reconstitution time of the same protein formulation prepared in the absence of a sugar and one or more amino acids, wherein the sugar is a disaccharide and is present in an amount from 1.0 to 20% w/v and wherein the one or more amino acids is present in an amount from or from above 50 mM to 200 mM.

9. The process according to claim 8, wherein the protein is an antibody or a fragment thereof.

10. The process according to claim 8, wherein the sugar is sucrose, trehalose or a mixture thereof.

11. The process according to claim 8, wherein the amino acid is glycine, L-proline, L-alanine, L-valine, L-serine, L-threonine, L-glutamine, L-asparagine, L-glutamate, L-aspartate, L-histidine, L-lysine, L-arginine or mixtures thereof.

12. The process according to claim 8, wherein the sugar is sucrose or trehalose and the amino acid is L-arginine hydrochloride, L-histidine hydrochloride, L-lysine hydrochloride or mixtures thereof.

13. The process according to claim 8, wherein the method comprises spray-drying a protein formulation further comprising a surfactant.

14. The process according to claim 13, wherein the surfactant is a polysorbate.

15. A protein formulation obtained through the process according to claim 8.

16. A method of therapy or diagnosis comprising administering the protein formulation of claim 15.

17. The method of claim 7, wherein the surfactant is polysorbate-20.

18. The method of claim 8, wherein the reconstituting comprising reconstituting with water.

19. The method of claim 14, wherein the surfactant is polysorbate-20.
